(19) United States
(12) Patent Application Publication
(10) Pub. No.: US 2010/0120097 A1
(43) Pub. Date: May 13, 2010

(54) METHODS AND COMPOSITIONS FOR NUCLEIC ACID SEQUENCING

(75) Inventors: Mikhail V. Matz, Austin, TX (US); Elisha Meyer, Austin, TX (US); Galina Aglyamova, Austin, TX (US); John K. Colbourne, Boomington, IN (US); Keithanne Mockaitis, Bloomington, IN (US); Jade Buchanan-Carter, Bloomington, IN (US)

Correspondence Address: CHALKER FLORES, LLP, 2711 LBJ FRWY, Suite 1036, DALLAS, TX, 75234, US

(73) Assignee: Board of Regents, The University of Texas System, Austin, TX (US)

(21) Appl. No.: 12/455,399

(22) Filed: Jun. 1, 2009

Related U.S. Application Data
(60) Provisional application No. 61/057,607, filed on May 30, 2008.

(51) Int. Cl. C12P 19/34

(52) U.S. Cl. 435/91.1

(57) ABSTRACT

The present invention includes novel compositions and method for nucleic acid sequencing. The methods and compositions permit a very large number of independent sequencing reactions to be arrayed in parallel, permitting simultaneous sequencing of a very large number of different oligonucleotides with superior output and quality.

### CROSS-REFERENCE TO RELATED APPLICATIONS

This application claims priority to U.S. Provisional Application Ser. No. 61/057,607, filed May 30, 2008, the entire contents of which are incorporated herein by reference.

### TECHNICAL FIELD OF THE INVENTION

The present invention relates in general to the field of nucleic acid sequencing.

### BACKGROUND OF THE INVENTION

Without limiting the scope of the invention, its background is described in connection with nucleic acid sequencing, and more particularly, improve methods and compositions for amplifying and determining nucleic acid sequences.

Since the discovery that nucleic acids encode the genome, it has been found that many diseases are associated with particular DNA sequences. Tremendous amounts of resources have been allocated to identify and correlate DNA sequence polymorphisms with a diseased state. These sequence polymorphisms include insertions, deletions, or substitutions of nucleotides in one sequence relative to a second sequence. As such, genome sequencing has become an increasing critical tool for diagnosis, therapy and prevention of illnesses and, eventually, the targeted modification of the human genome.

Development of rapid and sensitive nucleic acid sequencing methods utilizing automated DNA sequencers has revolutionized modern molecular biology. Analysis of entire genomes of plants, fungi, animals, bacteria, and viruses is now possible with a concerted effort by a series of machines and a team of technicians. Base sequencing of deoxyribonucleic acid and ribonucleic acid is one of the most important analytical techniques in biotechnology, the pharmaceutical industry, food industry, medical diagnostics and other fields of application.

Typically, a DNA sequence polymorphism analysis is performed by isolating DNA from an individual, manipulating the isolated DNA by digesting the DNA with restriction enzymes and/or amplifying a subset of sequences in the isolated DNA and examining the manipulated DNA. Commonly used procedures for analyzing DNA include electrophoretic-based separation analyses such as agarose or polyacrylamide gel electrophoresis. DNA sequences are typically inserted, or loaded on gels and subjected to an electric field. Because DNA has a uniform negative charge, DNA will migrate through the gel based on properties including sequence length and relative sizes.

Varieties of nucleic acid sequencing systems and methods have become available. For example, U.S. Pat. No. 5,972,693 provides methods by which biologically derived DNA sequences in a mixed sample or in an arrayed single sequence clone can be determined and classified without sequencing. The methods are based on the presence of carefully chosen target subsequences, typically 4 to 8 bases in length, in a sample DNA sequence together with DNA sequence databases containing lists of sequences likely to be present in the sample to determine a sample sequence. The method uses restriction endonucleases to recognize target subsequences to cut the sample sequence. Then, chosen recognition moieties are ligated to the cut fragments, the fragments are amplified, and the experimental observation made. Polymerase chain reaction (PCR) is the method of amplification. Several alternative embodiments were described which capable of increased discrimination and which use Type IIS restriction endonucleases, various capture moieties, or samples of specially synthesized cDNA. The '693 patent also uses information on the presence or absence of carefully chosen target subsequences in a single sequence clone together with DNA sequence databases to determine the clone sequence. Computer implemented methods are provided to analyze the experimental results and to determine the sample sequences in question and to carefully choose target subsequences to yield a maximum amount of information.

Another example can be found in the U.S. Pat. No. 6,190,868. Briefly, the patent discloses a methodology that provides positive confirmation that nucleic acids, possessing putatively identified sequence predicted to generate observed GeneCalling™ signals, are actually present within the sample from which the signal was originally derived. The putatively identified nucleic acid fragment within the sample possesses 3′- and 5′-ends with known terminal subsequences. The method in the '868 patent includes; contacting nucleic acid fragments in a sample in amplifying conditions with (i) a nucleic acid polymerase; (ii) “regular” primer oligonucleotides having sequences comprising hybridizable portions of the known terminal subsequences; and (iii) a “poisoning” oligonucleotide primer, the poisoning primer having a sequence comprising a first subsequence that is a portion of the sequence of one of the known terminal subsequences and a second subsequence that is a hybridizable portion of the putatively unidentified sequence which is adjacent to the one known terminal subsequence, where the nucleic acids amplified with the poisoning primer are distinguishable upon detection from nucleic acids amplified with the nucleic acids amplified only with the regular primers; separating the products of the contacting step; and detecting a sequence if the nucleic acids amplified with the poisoning primer are detected.

Yet another example can be found in the U.S. Pat. Nos. 6,274,380 and 7,211,390. Briefly, these patents disclose methods and apparatuses for sequencing a nucleic acid. The method includes annealing a population of circular nucleic acid molecules to a plurality of anchor primers linked to a solid support, and amplifying those members of the population of circular nucleic acid molecules which anneal to the target nucleic acid, and then sequencing the amplified molecules by detecting the presence of a sequence by-product.

The U.S. Pat. No. 7,244,567 teaches methods of sequencing both the sense and antisense strands of DNA with blocked and unblocked sequencing primers. These methods include the steps of annealing an unblocked primer to a first strand of nucleic acid; annealing a second blocked primer to a second strand of nucleic acid; elongating the nucleic acid along the first strand with a polymerase; terminating the first sequencing primer; deblocking the second primer; and elongating the nucleic acid along the second strand.

Yet another example is shown in the U.S. Pat. No. 7,335,762 to Rothberg et al. Briefly, Rothberg disclosed methods and apparatuses for sequencing a nucleic acid that permit a very large number of independent sequencing reactions to be arrayed in parallel, permitting simultaneous sequencing of a very large number (>10,000) of different oligonucleotides.

However, none of the above methods are adapted for high-throughput massively parallel sequencing. In particular, during sequencing of the protein-coding transcriptome, prior methods suffer from artifacts stemming from improper adapter ligation and/or primer annealing; do not provides even coverage of the length of individual transcripts; do not sequence efficiently, and some can not simultaneously sequence more than one sample.

### SUMMARY OF THE INVENTION

The present invention uses novel compositions to improve nucleic acid sequencing. The present invention dramatically reduces the fraction of unusable sequences corresponding to the adaptors in the total sequencing output, and eliminates artifacts due to improper adapter ligation and/or primer annealing. The present invention also provides even coverage of the length of individual transcripts due to strategic placement of the sequencing primer and improves the sequencing efficiency by pre-selecting the fragments with correct adapter combination. In addition, the present invention improves reliability and efficiency of the whole procedure due to reliance on PCR suppression rather than physical separation procedures. Furthermore, the present invention allows simultaneous sequencing of several samples.

In one aspect, the present invention provides methods and compositions for preparing a cDNA sample for sequencing. The steps include creating a double stranded cDNA by annealing a RNA having a poly A tail with a Cap-Trsa-CV oligonucleotide and reverse transcribing the RNA resulting in a full length double stranded cDNA; fragmenting (e.g., using sonication and/or nebulization) the full length double stranded cDNA to generate a plurality of fragmented double stranded cDNA; repairing the ends of the fragments using DNA polymerase (“end-polishing”), ligating a mixture of partially-double stranded A+ adapter and a partially-double stranded B+ adapter to fragmented double stranded DNA using a ligase; and amplifying the ligated double stranded cDNA using an amplification mixture comprising a primer A, a primer B, a A+-cap primer and a DNA polymerase.

In one aspect, the Cap-Trsa-CV oligonucleotide can include a cap primer sequence at the 5′ end and a broken poly T stretch region at the 3′ end. The broken poly T stretch region typically has two or more poly T regions about 6-base long separated by at least one base residue selected from dA, dC, and dG. This composition prevents pyrosequencing artifacts by eliminating the need to sequence through the long oligo dT stretch. The 3′-most base of the primer, is a mixture of dA, dC, and dG, to ensure that the primer initiates reverse transcription at the distal-most region of the polyA tail of the mRNA, rather than in the middle of it. In one aspect, the Cap-Trsa-CV oligonucleotide has the sequence listed in SEQ ID NO:1; the cap primer can have the sequence listed in SEQ ID NO. 2; and the A+-cap primer can have the sequence listed in SEQ. ID. NO: 3. In one aspect, the A+ adapter includes an A+ long oligonucleotide having a first suppression tag at the 3′ end and a A+ primer sequence at the 5′ end; and an A+ short oligonucleotide complementary to the first suppression tag. The suppression tag prevents amplification of fragments flanked by the same A+ adapter at both ends later in the procedure. The suppression tag of the A+ long oligonucleotide can be of different sequence and function as a barcode to identify the particular cDNA source post-sequencing.

In another aspect, the B+ adapter includes a B+ long oligonucleotide having a second suppression tag at the 3′ end and a B+ primer region at the 5′ end; and a B+ short oligonucleotide complementary to the second suppression tag. The suppression tag prevents amplification of fragments flanked by the same B+ adapter at both ends later in the procedure. The suppression tag of the B+ long oligonucleotide can be of different sequence and function as a barcode to identify the particular cDNA source post-sequencing. In one aspect, the step of step of ligation uses a molar ratio between about 0.9 to about 1.1 for the A+ adapter to B+ adapter, and the step of amplification uses a molar ratio of between about 0.9-1.1 to about 0.05-0.1 for the primer A:primer B to A+-cap primer.

The present invention also includes an A+ adapter and a B+ adapter oligonucleotides for amplification. Both adapters can further include a bar-coding tag (e.g., a biotin tag). The A+ adapter and a B+ adapter, each includes a long strand and an short strand and is capable of ligating to a first end or a second end of a fragmented double stranded cDNA. Typically, the long strand of the A+ adapter contains an A primer region at the 5′ end and a first suppression/barcode tag region at the 3′ end, and the long strand of the B+ adapter contains a B primer region at the 5′ end and a second suppression/barcode tag region at the 3′ end. Each of the first and second suppression tag regions prevents PCR amplification of the double stranded cDNA flanked with the same A+ adapter or the same B+ adapter at both ends. Only the double stranded cDNA fragments with both A+ and B+ adapters are capable of being amplified.

In some aspects, the primer cocktail includes an A primer, B primer, and A+-cap primer. In another aspect, the long strand A+ adapter can have the sequence listed in SEQ ID NO: 4; the short strand A+ adapter can have the sequence listed in SEQ ID NO: 5; the long strand B+ adapter can have the sequence listed in SEQ ID NO: 6; and the short strand B+ adapter can have the sequence listed in SEQ ID NO: 7.

In another aspect, the molar ratios of the A+ adapter:B+ adapter during the ligation step comprises about 0.9 to 1.1:about 0.9 to 1.1, and the molar ratio of A primer, B primer, and A+-cap primer comprises about 0.9 to 1.1:about 0.9 to 1.1:about 0.04 to 0.11.

### BRIEF DESCRIPTION OF THE DRAWINGS

For a more complete understanding of the features and advantages of the present invention, reference is now made to the detailed description of the invention along with the accompanying figures and in which:

FIG. 1 is a schematic diagram of the present invention.

FIG. 2 shows the preparation of a cDNA sample for 454 Sequencing.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

### Example 1

In one embodiment, the present invention describes method to prepare cDNA samples for sequencing, for example, a 454 Sequencing™ known by the skilled artisan. A 454 Sequencing™ is a parallel pyrosequencing system capable of sequencing about 100 megabases of raw DNA per run. The system relies on fixing nebulized and adapter-ligated DNA fragments to small DNA-capture beads in a water-in-oil emulsion. The DNA fixed to these beads is then amplified by polymerase chain reactions (PCR). Finally, each DNA-bound bead is placed into a approximately 44 μm well on a PicoTiterPlate fiber optic chip for sequencing.

In the 454 Sequencing™ protocol, four nucleotides are typically washed in series over the PicoTiterPlate. During the nucleotide flow, each of the beads with millions of copies of DNA is sequenced in parallel. If a nucleotide complementary to the template strand is flowed into a well, the polymerase extends the existing DNA strand by adding nucleotides. Addition of one or more nucleotides results in a reaction that generates a light signal that is recorded by the CCD camera in the instrument. This technique is also called pyrosequencing. The signal strength is proportional to the number of nucleotides.

In 454 Sequencing™, genomic DNA is typically broken down into 300-500 base pairs smaller fragments and are subsequently “polished” (blunted). Short adaptors are ligated onto the ends of the fragments. These adaptors provide priming sequences for both amplification and sequencing of the sample-library fragments. Typically, one adaptor can contain a 5′-biotin tag that enables immobilization of the library onto streptavidin coated beads. After nick repair, the non-biotinylated strand is released and used as a single-stranded template DNA (sstDNA) library. The sstDNA library is assessed for its quality and the optimal amount (DNA copies per bead) needed for emPCR is determined by titration.

The sstDNA library is immobilized onto beads. The beads containing a library fragment carry a single sstDNA molecule. The bead-bound library is emulsified with the amplification reagents in a water-in-oil mixture. Each bead is captured within its own microreactor where PCR amplification occurs. This results in bead-immobilized, clonally amplified DNA fragments.

The present invention illustrates methods and compositions for preparation of cDNA samples for sequencing. The present invention enables the sequencing process to avoid repeated unproductive sequencing of adaptor regions, generates significantly less artifactual sequences stemming from improper adapter ligation and/or primer annealing; it provides even coverage of the length of individual transcripts due to strategic placement of the sequencing primer; it improves the sequencing efficiency by pre-selecting the fragments with correct adapter combination; it improves reliability and efficiency of the whole procedure due to reliance on PCR suppression rather than physical separation procedures; and it allows simultaneous sequencing of several samples.

In certain embodiments, the present invention can be used for new-generation sequencing using pyrosequencing. An example can be found in the publication by Margulies M, Egholm M, Altman W E, Attiya S, Bader J S, et al. (2005) Genome sequencing in microfabricated high-density picolitre reactors. Nature 437: 376-380.

The initial cDNA is produced using SMART cDNA amplification kit described in Zhu et al., but with different cDNA synthesis primer: Cap-Trsa-CV (first strand cDNA synthesis primer): 5′-AAGCAGTGGTATCAACGCAGAGT CGCAGTCGGTACTTTTTTCTTTTTTV-3′ (SEQ. ID NO: 1) The 5′ end includes a “cap” primer sequence 5′-AAGCAGTGGTATCAACGCAGAGT-3′ (SEQ. ID NO: 2), and the 3′ end includes a “broken chain” poly T region. The portion of the Cap-Trsa-CV primer in between the cap primer sequence and polyT stretch can be variable or absent. Alternatively, the cDNA can be amplified by any other method or non-amplified double-stranded cDNA can be used, as long as its synthesis incorporates the Cap-Trsa-CV primer.

The purpose of the “broken chain” T-primer is to reduce read artifacts during pyrosequening, which may be thrown out of calibration by a too strong signal produced from a long mononucleotide stretch (such as polyT or polyA).

The cDNA may be optionally normalized using Trimmer kit and re-amplified using “cap” primer; nebulized and/or sonicated to the average fragment size of 350-400 base pairs; end-polished (by incubation with a DNA polymerase and dNTPs in appropriate buffer) and ligated to the mixture of “A+” and “B+” adapters.

Each of these adapters is an equal molar mixture of two oligos (typically, 1 μM each in the working concentration), a long one that actually gets ligated by its 3′ end, and a short one that complements to the 3′ end of the longer one to mimic the double-stranded blunt end for the ligase. The short oligo is not getting ligated since it does not have a 5′-phosphate.

The A+ adapter also includes a long and a short strand oligo. The long strand oligo:

5′-GCCTCCCTCGCGCCATCAG CCGCGCAGGT-3′ (SEQ. ID NO: 4) has an A primer sequence at the 5′ end and a suppression/barcoding tag at the 3′ end. The Short oligo has the sequence 5′-ACCTGCGCGG-3′ (SEQ. ID NO: 5), complementary to the suppression/barcoding tag of the long oligo.

The B+ adapter includes a long and a short strand oligo. The long strand oligo:

5′-GCCTTGCCAGCCCGCTCAG ACGAGCGGCCA-3′ (SEQ. ID NO: 6) has a B primer sequence at the 5′ end and another suppression/barcoding tag at the 3′ end. The short oligo has the sequence 5′-TGGCCGCTCGT-3′ (SEQ. ID NO: 7), complementary to the suppression/barcoding tag of the long oligo.

It is important to note that the adapters typically only get ligated to the “new” 5′ ends formed as a result of fragmentation/polishing, since the original 5′ termini correspond to the incorporated “cap” primer used for amplification and don't bear the 5′ phosphates.

The product of ligation is then amplified using a mixture of three primers: A and B primers in 0.1 μM concentration (their sequence was incorporated into the ligated adaptors) and a long “step-out” primer (“A+-cap”, in the typical concentration of about 0.005-0.01 uM) that allows the A+ sequence to get attached to the original cDNA termini.

The A+-cap primer has the sequence:

5′-GCCTCCCTCGCGCCATCAG CCGCGCAGGTAAGCAGTGGTATCAACGCAGAGT-3′ (SEQ. ID NO: 3) with an A primer sequence at the 5′ end, a suppression tag in the middle, and a cap primer sequence at the 3′ end.

During this amplification “suppression tags” invoke PCR suppression effect for the fragments that end up flanked by the same kind of adapter, which results in exclusive amplification of the fragments flanked by both A and B primers. In these fragments, B primer is found only on the “inside” of the original cDNA sequence (i.e., fragmentation points introduced during sonication and/or nebulization) while A primer can be either inside (by virtue of adaptor ligation) or “outside”, i.e. flanking the original cDNA termini (by virtue of step-out amplification).

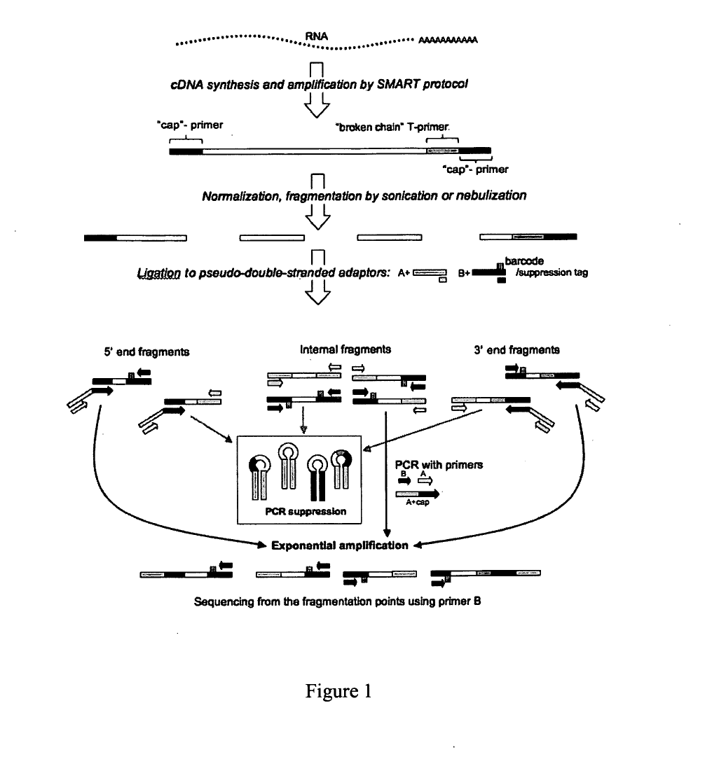

The entire step is summarized in an example schematic diagram shown in FIG. 1. In FIG. 1, a biotinylated A primer can be used to bind the fragments to beads and B primer can be a sequencing one. The suppression/barcoding tag of B primer can be variable and can used to discriminate samples that are sequenced simultaneously in the same plate. The barcode sequence can also be incorporated into A+ adapter and/or A+ cap primer.

The method disclosed herein does not suffer from problems associated with improper adapter ligation or primer annealing and improves sequencing efficiency by eliminating the fragments with incorrect adapters (same kind of adapters on both ends). Modification of the cDNA synthesis procedure avoids incorporation of long dT-stretches originating from the polyA tails of the mRNA, which otherwise would create problems during pyrosequencing stage. cDNA fragments made with the methods and compositions disclosed herein bear the sequencing primer only on the ends corresponding to the fragmentation sites of the original mRNAs rather than 5′ or 3′ termini, thus ensuring even coverage of the mRNA and efficient assembly and dramatically reducing the ballast fraction of total sequence output corresponding to 3′ and 5′-adaptor regions. cDNA samples can be “barcoded” by different adaptors and processed together in the same sequencing run.

Application of the present invention includes transcriptome sequencing de novo or transcriptome re-sequencing. Other applications include genetic marker discovery and profiling, gene expression analysis, molecular identification of unknown samples, environmental genomics.

The present inventors demonstrated the surprising and unexpected results obtained using the procedure of the present invention by constructing two normalized cDNA libraries: from larvae of coral Acropora millepora and from adult amphipod crustaceans Hyallela sp., followed by sequencing using Roche 454 FLX system. The cDNA preparations procedure from the present invention results consistently in the number of reads exceeding the published transcriptome-sequencing studies by a factor of two or more, and show a remarkable improvement in the fraction of usable reads (i.e., sufficiently long high-quality pyrosequencing readouts with no polyA runs) (Table 1) Table 1 shows the comparison of the gross outputs of de novo transcriptome sequencing.

TABLE 1

| | | # reads per | |
|---|---|---|---|
| Method | organism | run | % usable |
| Present invention | coral Acropora millepora | 628649 | 99.0 |
| Present invention | amphipod Hyallela | 626623 | 97.1 |
| Vera et al, 2008 | butterfly Melitaea cinxia | 304027 | 85.2 |
| Weber et al, 2007 | mustard weed Arabidopsis | 277663 | 86.5 |
| | thaliana | | |

### Example 2

Preparation of cDNA samples for de novo transcriptome sequencing with 454 technology. The preparation of appropriately modified cDNA is a critical step ensuring the overall success of transcriptome diversity characterization using next-generation sequencing methods. Example 2 is method that has been adapted for the use with 454 technology, with the primary focus on protein-coding transcriptome data assembly and annotation de novo (i.e., in the absence of the reference genome data). This method generates pools of fragmented cDNAs flanked by two standard 454 amplification/sequencing primers, ready for amplification of individual sequences on microbeads and sequencing. The method requires as little as 50 ng total RNA at the start, and solves three most important problems inherent in comparable protocols: artifacts due to long A/T homopolymer regions, large proportion of unusable (adaptor) sequences in the 454 output, and coverage bias towards 3′-termini of transcripts.

The developed method uses PCR-suppression effect to eliminate problems associated with improper adapter ligation, primer annealing, and adaptor concatenation. Modification of the cDNA synthesis procedure avoids incorporation of long A/T-stretches originating from the polyA tails of the mRNA, which would create problems during pyrosequencing stage. cDNA fragments in samples produced by this method bear the sequencing primer only on the ends corresponding to the fragmentation sites of the original mRNAs rather than 5′ or 3′ termini, facilitating even coverage and further lowering the proportion of unusable adaptor sequences in the output. To further reduce the 3′-end bias, the method uses two approaches. First, the desired distribution of lengths within the originally produced cDNA can be achieved by varying the conditions of the amplification reaction (there is no physical separation procedure involved). Second, the final product is generated as three separate samples, specific to 3′-terminal, 5′-terminal, and middle cDNA fragments, which can be then mixed in a desired proportion or sequenced independently. To enable simultaneous sequencing of several samples, the method uses its own cDNA barcodes incorporated into adaptor sequences.

The present invention includes the following advantages: (1) requires small amount of total RNA as a staring material; (2) high output of useful sequence due to elimination of adaptor-related artifacts (2-5 fold more new sequence data per run than in analogous published applications); (3) provides even coverage of the length of individual transcripts due to strategic placement of the sequencing primer and production of separate samples for 5′, 3′, and middle cDNA fragments; (4) eliminates the need for strand-selection step prior to emulsion PCR due to the inherent control over adaptor configurations; and (5) allows simultaneous sequencing of several samples through adaptor barcoding.

The initial cDNA is produced using SMART cDNA amplification kit (Clontech) (Zhu et al, 2001) but with different cDNA synthesis primer: Cap-Trsa-CV (first strand cDNA synthesis primer):

(SEQ. ID NO: 8)
5′- AAGCAGTGGTATCAACGCAGAGT CGCAGTCGGTACTTTTTTCTTTTTTV -3′
(“cap” primer sequence) (“broken chain” polyT)

The purpose of the “broken chain” T-primer is to reduce read artifacts during 454 pyrosequening, which may get thrown out of calibration by a too strong signal produced from a long mononucleotide stretch (such as polyT or polyA).

The cDNA is then: [optionally] normalized using Trimmer kit (Evrogen) and re-amplified using cap primer; nebulized or sonicated to the average fragment size of 500-1000; and end-polished (by incubation with a DNA polymerase and dNTPs in appropriate buffer) and ligated to the mixture of “Atitn+” and “Btitn+” adapters.

Each of these adapters is an equimolar mixture of two oligos (typically, 1 uM each in the working concentration), a long one that actually gets ligated by its 3′ end and a short one that complements to the 3′ end of the longer one to mimic the double-stranded blunt end for the ligase. The short oligo is not getting ligated since it does not have a 5′-phosphate.

(SEQ. ID NO: 9)

Atitn + adapter:
Long oligo:
5′-TCCCTGCGTGTCTCCGACTCAG CCGCGCAGGT -3′
Atitn primer sequence suppression tag + barcode
(underlined)

(SEQ. ID NO: 10)

Short oligo:
5′- ACCTGCGCGG -3′

This one has a CAG barcode. Here are some other possible variants of barcoded Atitn+ adaptors (pairs of long and short oligos):

(SEQ. ID NO: 11)

| | |
|---|---|
| GAC: | TCCCTGCGTGTCTCCGACTCAG CCGCGGACGT |
| | ACGTCCGCGG |

(SEQ. ID NO: 12)

| | |
|---|---|
| AGC: | TCCCTGCGTGTCTCCGACTCAG CCGCGAGCGT |
| | ACGCTCGCGG |

(SEQ. ID NO: 13)

| | |
|---|---|
| CGA: | TCCCTGCGTGTCTCCGACTCAG CCGCGCGAGT |
| | ACTCGCGCGG |

(SEQ. ID NO: 14)

| | |
|---|---|
| ACG: | TCCCTGCGTGTCTCCGACTCAG CCGCGACGGT |
| | ACCGTCGCGG |

(SEQ. ID NO: 15)

| | |
|---|---|
| GCA: | TCCCTGCGTGTCTCCGACTCAG CCGCGGCAGT |
| | ACTGCCGCGG |

(SEQ. ID NO: 16)

| | |
|---|---|
| CTG: | TCCCTGCGTGTCTCCGACTCAG CCGCGCTGGT |
| | ACCAGCGCGG |

(SEQ. ID NO: 17)

| | |
|---|---|
| CGT: | TCCCTGCGTGTCTCCGACTCAG CCGCGCGTGT |
| | ACACGCGCGG |

(SEQ. ID NO: 18)

| | |
|---|---|
| GTC: | TCCCTGCGTGTCTCCGACTCAG CCGCGGTCGT |
| | ACGACCGCGG |

(SEQ. ID NO: 19)

| | |
|---|---|
| GCT: | TCCCTGCGTGTCTCCGACTCAG CCGCGGCTGT |
| | ACAGCCGCGG |

Btitn + adapter:

(SEQ. ID NO: 20)

| | |
|---|---|
| Long | 5′- TGTGTGCCTTGGCAGTCTCAG ACGAGCGGCCA -3′ |
| oligo: | Btitn primer sequence suppression tag |

(SEQ. ID NO: 21)

| | |
|---|---|
| Short | 5′- TGGCCGCTCGT -3′ |
| oligo: | |

It is important to note that the adapters only get ligated to the “new” 5′ ends formed as a result of fragmentation/polishing, since the original 5′ termini correspond to the incorporated “cap” primer used for amplification and don't bear the 5′ phosphates.

The protocol allows for independent amplification of fragment pools corresponding to 5′-ends, internal fragments and 3′-ends of the original cDNAs. These pools may be then either sequenced separately or mixed in a desired proportion to ensure even coverage. In particular, 5′-end samples are enriched with coding sequences and are especially useful for obtaining pilot gene hunting or phylogenetics data.

Three different primer combinations are used to amplify different cDNA ends. 3′-ends are amplified with Atitn and Btitn+TrsaC primers, internal fragments—with Atitn and Btitn, 5′-ends—with Atitn and Btitn+halfswitch (see below for primer sequences). All primers are typically used in 0.1 uM concentration.

Atitn primer:
(SEQ. ID NO: 22)

5′- TCCCTGCGTGTCTCCGACTCAG-3′
Btitn primer:

(SEQ. ID NO: 23)
5′-TGTGTGCCTTGGCAGTCTCAG-3′
Btitn + halfswitch primer:
(SEQ. ID NO: 24)
5′- TGTGTGCCTTGGCAGTCTCAG ACGAGCGGCCA GTATCAACGCAGAGTACATGG -3′
(Btitn primer sequence) (suppression tag) (sequence of the 3′-portion of the
template-switch oligo)
Btitn + TrsaC:
(SEQ. ID NO: 25)
5′- TGTGTGCCTTGGCAGTCTCAG ACGAGCGGCCA CGCAGTCGGTACTTTTTTCTTTTTT
(Btitn primer sequence) (suppression tag) (sequence of the 3′-portion of the
“broken chain” cDNA synthesis primer)

During this amplification “suppression tags” invoke PCR suppression effect for the fragments that end up flanked by the same kind of adapter, which will results in exclusive amplification of the fragments flanked by both Atitn and Btitn primers. In these fragments Atitn primer is found only on the “inside” of the original cDNA sequence (i.e., fragmentation points introduced during sonication or nebulization) while Btitn primers can be either inside (by virtue of adaptor ligation) or “outside”, i.e. flanking the original cDNA termini (by virtue of step-out amplification). Such strategic positioning of the sequencing primer (Atitn) in the final sample eliminates the need for strand-selection step prior to emulsion PCR and further improves the evenness of coverage. As the last stage of the protocol, the products of amplification corresponding to the size range 500-1000 by are purified from the agarose gel.

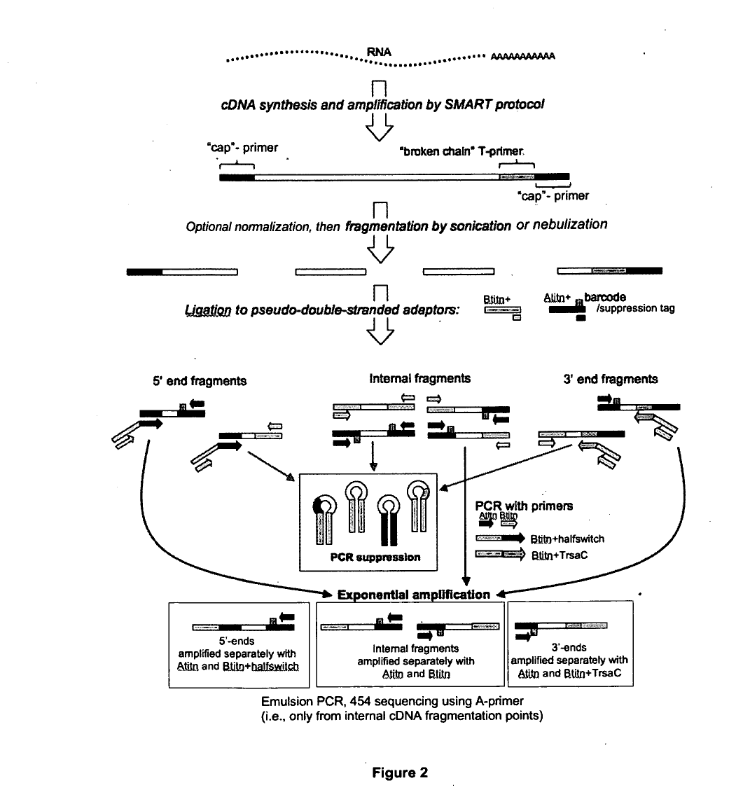

The following detailed protocol outlines the basic steps of the present invention as outlined in FIG. 2.

RNA template preparation. These steps are recommended but may not be necessary, depending on your protocol of choice for isolating total RNA. Begin with about 0.5-1 μg RNA from the organism of your choice (note: the latest version of the Clontech's SMART kit claims the amount can be as low as 50 ng). Precipitate RNA by adding 1 volume 13.3 M LiCl, incubating 30 minutes at −20° C., and centrifuging 20 minutes at 16g at room temperature. Rinse RNA pellets briefly with 80% ethanol (don't centrifugate), air dry at room temperature, and dissolve pellets in EB (10 mM Tris, pH 8.0).

Analyze RNA on a gel to evaluate integrity. First-strand cDNA synthesis (at this and the next stage, follow Clontech's SMART cDNA amplification protocol, but replace the cDNA synthesis primer by Cap-TRSA-CV).
1. Combine 4 μl RNA (for a total of 1000 ng RNA) with 1 μl 10 μM Cap-TRSA-CV primer. Incubate 3 minutes at 65° C., then chill on ice.
2. To the above tube, add a premixed solution containing the following: 2 μl 5× first-strand synthesis buffer; 0.5 μl 10 mM dNTP; 1 μl 0.1 μM DTT; 1 μl 10 μM template-switch primer (provided with the Clontech's kit); 1 μl Superscript II reverse transcriptase (Invitrogen).
3. Incubate at 42° C. for 1 hour.
4. Terminate the reaction by incubating at 65° C. for 15 minutes, then return tube to ice.
5. Dilute 5-fold in water to minimize carryover of primers into subsequent reactions.

cDNA amplification. For each first-strand-cDNA sample, set up 12 PCR reactions (30 μl each): 3 μl diluted FS-cDNA (from step 2e); 21 μl H₂O; 3 μl 10×PCR buffer; 0.75 μl 10 mM dNTP; 1.4 μl 10 μM cDNA amplification primer (from Clontech's SMART cDNA amplification kit); 0.6 μl Advantage2 polymerase (Clontech).

Optional: use 1.5 μl of 10 μl Lu4sCap primer for amplification instead of the primer supplied in the Clontech kit to obtain higher molecular weight product (>1.5-2 kb), due to mild PCR-suppression effect. Lu4sCap primer:

(SEQ. ID NO: 26)

5′-AGTGGACTATCCATGAACGCAAAGCAGTGGTATCAACGCAGAGT-3′

1. Amplify using the following profile:
2. 94° C. for 5 minutes;
3. (94° C. for 40 seconds, 65° C. for 1 minute, 720 C for 6 minutes)×(15-19) cycles depending on the sample
4. (Lu4sCap primer may require more cycles, up to 25).
5. After PCR, hold product at room temperature.
6. Evaluate PCR product by loading 3 μl on a gel and visualizing with ethidium bromide. There should be a faintly visible smear with some bands, with the majority of product falling between 500 and 3000 by in length. Add 3 more cycles if there is nothing visible on the gel, then evaluate again. If the product is not amplified in 20 cycles (25 for Lu4sCap), something is wrong—start over from the cDNA synthesis step. NOTE: the total amount of cDNA product per tube should not exceed 200 ng, which means that the smear on the agarose gel (20 ng per lane) should be really faint. Make sure you don't over-amplify cDNA beyond that.
7. To maximize the amount of PCR product that is double-stranded, “chase” the reactions by adding the original amount of primer again (1.4 μl of 10 μM cDNA amplification primer) and cycling with the following profile:
8. 78° C. for 1 minute, 65° C. for 1 minute, 720 C for 7 minutes.
9. Combine together 12 separate reactions prepared from each first-strand-cDNA sample, and purify this PCR product on a column (we use Qiagen Qiaquick PCR Purification kit). Elute the final sample in 50-100 μl of EB (10 mM tris-HCl pH 8.0). Measure the concentration of DNA using Nanodrop spectrofluorometer or any other appropriate method; there should be at least 2 μg of DNA in total. Then, go directly to Sonication (step 6) or do optional Normalization step.

Normalization (Optional)
1. EtOH precipitate the product to concentrate (i.e. if the resulting concentration is less than 2 μg in 12 microliters) and dissolve it appropriate volume of miliQ water (but don't use water to elute DNA from the column on previous step!) 5 μl ( 1/10 volume) of 3M NaAcetate pH 4.8-5.2; 125 μl (2.5 volume) 96-100% EtOH; hold 20-30 minutes at −20° C.; Spin 20 minutes at maximum speed at 4° C., rinse the with 70% EtOH, air dry, dissolve in appropriate volume of milliQ water to achieve a concentration of 2 μg in 12 microliters.
2. The Trimmer kit from Evrogen is used essentially according to the manufacturer's instructions, here we are just replicating their protocol. Prepare a hybridization master mix by combining: 2 μg cDNA from step 3f in ≦12 μl volume; 4 μl 4× hybridization buffer; H₂O to a total volume of 16 μl; (Note that final cDNA concentration=125 ng μl⁻¹)
3. Aliquot this out into 4 individual PCR tubes (4 μl each) and overlay each with a drop of sterile mineral oil; centrifuge briefly to collect liquid and separate phases.
4. Using a thermal cycler, incubate at 98° C. for 2 minutes, then at 68° C. for 5 hours, then proceed immediately to the next step.
5. Near the end of the hybridization period (step 4d), warm the DSN master buffer (Trimmer kit) to 68° C.
6. Prepare a ½ and ¼ strength dilutions of the double-strand specific nuclease (DSN) using DSN storage buffer as the diluent; store on ice until ready to use.
7. At the end of the hybridization period, add 5 μl preheated master buffer to each tube. Spin briefly in a bench-top centrifuge and return immediately to the thermal cycler. It is important to maintain the temperature at 68° C. during this period, so minimize time spent out of the thermal cycler (no more than a few seconds).
8. To the four tubes from step 4c, add the following, while maintaining temperature:

| Tube | Add |
|---|---|
| A | 1 μl un-diluted DSN enzyme |
| B | 1 μl ½ dilution DSN enzyme |
| C | 1 μl ¼ dilution DSN enzyme |
| D | 1 μl DSN storage buffer (diluent) |

10. Incubate at 68° C. for 25 minutes.
11. Add 10 μl of DSN stop solution (Trimmer kit) to each tube, mix well, and spin briefly to collect contents.
12. Incubate at 68° C. an additional 5 minutes.
13. Add H2O to each tube then store at −20° C. or proceed with next steps.

Amplification of Normalized cDNA
1. Set up 4 separate PCR reactions, each containing: 1 μl diluted normalized cDNA (from step 4l), one PCR reaction per DSN treatment; 23 μl H₂O; 3 μl 10×PCR buffer; 0.75 μl 10 mM dNTP; 1.4 μl 10 μM cDNA amplification primer (from Clontech's SMART cDNA amplification kit); 0.6 μl Advantage2 polymerase (Clontech)
2. Amplify using the following profile: 94° C. for 5 minutes; (94° C. for 40 seconds, 65° C. for 1 minute, 72° C. for 6 minutes)×5 cycles.
3. Remove all tubes from thermal cycler. Remove a 5-μl aliquot from the control tube (corresponding to template tube D, in step 4h) and set this aside.
4. Amplify the control tube for an additional 2 cycles (total=7). Remove another 5-μl aliquot and set aside.
5. Repeat step 5d twice more, producing aliquots from this tube that correspond to 5, 7, 9 and 11 cycles.
6. Load all aliquots from step 5e on a gel to evaluate optimum cycle number X as described in the manufacturer's instructions (for our experiments, X=6).
7. Return DSN-containing reactions to the thermal cycler and amplify for an additional N cycles, where N=X+9−5 (for our experiments, X+9−5=8, for 15 cycles total in experimental tubes).
8. “Chase” all reactions as described in step 3d.
9. Load 5 μl on a gel to determine which enzyme dilution treatment (1, ½, or ¼) gave the best results, as described in Trimmer kit instructions.
10. Once both the optimum cycle number (step 5g) and the optimum enzyme treatment (step 5i) have been established, prepare 16 individual 30 μl reactions according to those treatments and repeat steps 5a-i. Again, avoid over-amplifying the cDNA (see note at the step 3c).
11. Pool the products, purify on a column (e.g., Qiagen Qiaquick), elute in EB, and quantify. Normalized cDNA can be stored at −20° C.

Fragmentation (sonication). In certain circumstances it sonication can be used to nebulize the fragments since it makes it easier to process multiple samples at once, and poses less threat of DNA contamination. Sonication was conducted with a “cup horn” attachment: a water-filled cup with sonicating bottom in which the 1.5 mL tubes may be submerged. Our model is called “ultrasonic liquid processor Sonicator 3000” by Misonix, with cup horn part number 431 C.
1. Prepare a tube of normalized (optional), amplified, purified cDNA (from step 3e or 5k) containing ˜1-5 μg cDNA in 100 μl Dilute with EB if required to achieve this concentration (˜50 ng/μl).
2. Set aside an aliquot of intact cDNA at this time for later gel analysis.
3. Set up a sonicator with an ice water bath so that a 1.5-ml centrifuge tube can be partially submerged in the water, with the bottom of the tube resting ˜1 cm above the cup horn bottom, and the portion of that tube containing liquid fully submerged in the water.
4. Set the sonicator power at 1.0-1.5, corresponding to 18-30 W.
5. Sonication should be done in 30 second “on” bursts, with 30 second “off” rests in between. Note that sonication times are reported here as the sum of all “on” periods during the process.
6. Sonicate the cDNA for a series of increasing durations, and remove an aliquot at each interval. In our experiments, we choose 1 minute, 3 minutes, 5 minutes, 7 and up to 10 minutes.
7. After all sonication is complete, load 2-3 μl of each sample (including the original intact cDNA) on a gel to evaluate the molecular weight. Select the treatment that produced a smear ranging from about 500 to about 2000 bp. In our experiments, this is commonly the 7-9 minute treatment.
8. Precipitate the fragmented cDNA with ethanol to remove very short oligonucleotides, and dissolve in 10-20 μl of a suitable buffer (EB or 1×NEB2).

Polishing and Ligation with Adaptors.
1. Polish the fragmented cDNA to ensure that all ends are blunted, by combining the following in a tube at room temperature: 25 ng fragmented cDNA (from step 6h); 1.25 μl 10×NEB2 buffer; 1.25 μl 10×BSA; 0.6 μl 10 mM dNTP; 0.6 μl T4 DNA polymerase; 0.6 μl Klenow fragment of DNA polymerase I (New England Biolabs or equivalent); H₂O to final volume=12.5 μl
2. Incubate at room temperature for 1½ hours.
3. Terminate polishing reaction by incubating at 70° C. for 15 minutes, then cool to room temperature.
4. Prepare adaptor Atitn by combining Atitn+ barcoded primer and anti-Atitn+barcoded primer at a final concentration of 10 μM each. Do the same mix for Btitn+ and antiBtitn+ at a final concentration 10 μM each.
5. Prepare ligation master mixes at room temperature by combining: 5 μl H2O; 1.25 μl 10× T4 DNA ligase buffer; 2.5 μl 10 μM adaptor Atitn+(bar-coded); 2.5 μl 10 μM adaptor Btitn+; 1.25 μl T4 DNA ligase
6. Combine 12.5 μl master mix with 12.5 μl polished cDNA (from step 7c) for a final volume of 25 μl.
7. Incubate at 12° C. overnight.
8. The following day incubate ligation mix 10 minutes at 65° C., then cool to room temperature—do not store on ice.
9. Purify on a column (e.g., Qiagen Qiaquick) according to the manufacturer's instructions, and elute in 30 μl EB.

PCR Testing the Ligation.
1. For each 454 cDNA library produced, prepare 5 different PCR reactions, each with a different combination of primers. Including water controls (no-template controls) is recommended. The primer combinations are as follows (final primer concentrations are shown):

| Tube | Primers |
|---|---|
| 1 | 0.2 μM Atitn |
| 2 | 0.2 μM Btitn |
| 3 | 0.1 μM Atitn, 0.1 μM Btitn |
| 4 | 0.1 μM Atitn, 0.1 μM Btitn + halfswitch, |
| 5 | 0.1 μM Atitn, 0.1 μM Btitn + TrsaC |

The reactions 3, 4, and 5 specifically amplify internal fragments, 5′-ends, and 3′-ends of the original cDNAs, respectively.
2. Each PCR reaction is assembled as follows: 3 μl 10×PCR buffer; 0.75 μl 10 mM dNTP; 1 μl ligation product (from Polishing and Ligation step 9); 0.6 μl Advantage2 polymerase; Primers from Step 1 of the PCR Testing and Ligation); H₂O to final volume of 30 μl
3. Amplify these reactions using the following profile: 94° C. for 5 minutes; (94° C. for 40 seconds, 65° C. for 1 minute, 72° C. for 1 minutes)×17 cycles; a typical targeted product is the 500 bp-1000 bp length 1 minute of elongation time is enough.
4. Load 3 μl of these products on a gel; hold remainder at room temperature while the gel runs in case additional cycles are required.
5. A visible smear ranging from 300-2000 by should be visible in reaction #3, #4 and #5. None of the other two reactions should produce any product.
6. If nothing is visible in any lanes, amplify for an additional 2 cycles and repeat the gel analysis.
7. Repeat previous step until visible smears are produced to allow determination of optimum cycle number. If more than 17 cycles were required to produce visible smears, try adding more template and using fewer cycles. In our experiments, 1 μl of purified ligation product as PCR template and 17 cycles produced visible smears based on loading 3 μl on a gel.

Amplification of Samples for Gel Extraction
1. Set up “bulk” amplifications based on the optimum cycle numbers and template volumes determined from the PCR tests above (steps 8a-g). For our experiments, we set up 8 reactions.
2. Each reaction is assembled as follows: 3 μl 10×PCR buffer; 0.75 μl 10 mM dNTP; X μl ligation product (determined in PCR testing the ligation steps 1-6); 0.5 μl 6 μM primer Atitn; 0.5 μl 6 μM primer Btitn OR Btitn+halfswitch OR Btitn+TrsaC; 0.6 μl Advantage2 polymerase; H2O to final volume of 30 μl
3. Amplify the reactions using the following profile:
4. 94° C. for 5 minutes;
5. (94° C. for 40 seconds, 65° C. 1 minute, 72° C. for 1 minutes) X N cycles;
6. where N is the optimum cycle number determined (8a-g).
7. Load an aliquot on a gel to verify that the reaction amplified as expected.
8. Chase using the following profile:
9. 78° C. 1 minute, 65° C. 1 minutes, 68° C. 1 minutes.
10. PCR purify using a column (e.g., Qiagen Qiaquick), elute in 30 μl EB, and quantify.

Gel purification of final samples. For Titanium 454 procedure it is extremely important to have DNA fragments within 500-1000 by length range. The following protocol is a modified version of a standard agarose electrophoresis, which improves separation due to the buffer concentration gradient forming in the gel.
1. Make a 1% agarose gel using SeaKem GTG Agarose (Lonza 50071)
2. Put the gel in the apparatus, pour 1×TBE buffer in the “lower” (cathode) chamber and 0.5× TBE buffer (TBE buffer diluted twice with water) into the “upper” (anode) chamber. Take care not to mix the buffers; the buffer should not cover the top, of the gel. Wash the wells with 1×TBE. Pre-run the gel for 10 minutes at 100V.
3. Load all DNA from step 9f combined with 6× loading dye. It will be 3 kinds of samples: 5′ ends, 3′ ends and the “middles”. For each of them you might need to do more than one gel-load as the usual amount of DNA extracted from the gel is around 200 ng per 1 cm-wide lane, and you want to get 1 μg of material total in the end.
4. Run at 100V for 1 hour 15 minutes or optimum time.
5. Cut the pieces of gel with the smear between 500 bp and 1000 bp, avoiding the edges of the lane. Note: ethidium bromide in the buffer and the gel may be used, and view the gel on UV-transilluminator; but any appropriate staining/visualizing method can be used. In you are using UV, minimize exposure of the gel to UV during cutting to avoid damaging your samples.
6. Extract the DNA from the gel. We used QIAEX II Gel extraction Kit. (Qiagen, 20021). At the last step, elute in smaller volume of 10 mM TRIS or EB buffer. We used 15 μl+15 μl for the total volume 20 μl. Then spin one more time to clean the eluate form any residue DNA-binding beads. That allows getting higher concentration and more precise reading on the nanodrop spectrophotometer.
7. Quantify it and mix in desirable proportions (or keep separate).

Now the sample is ready for 454. NOTE: For use in the 454 process, it is best if the final cDNA sample is tested to confirm that is free from artifacts. Ligate an aliquote of it into any PCR-cloning vector (such as pGEM-T, Promega) and sequence 10-20 randomly picked clones using standard Sanger technique.

It is contemplated that any embodiment discussed in this specification can be implemented with respect to any method, kit, reagent, or composition of the invention, and vice versa. Furthermore, compositions of the invention can be used to achieve methods of the invention. It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

Vera J C, Wheat C W, Fescemyer H W, Frilander N U, Crawford D L, et al. (2008) Rapid transcriptome characterization for a nonmodel organism using 454 pyrosequencing. Molecular Ecology 17: 1636-1647.
Weber A P M, Weber K L, Can K, Wilkerson C, Ohlrogge J B (2007) Sampling the arabidopsis transcriptome with massively parallel pyrosequencing. Plant Physiology 144: 32-42.
Zhu Y Y, Machleder E M, Chenchik A, Li R, Siebert P D (2001) Reverse transcriptase template switching: A SMART (TM) approach for full-length cDNA library construction. Biotechniques 30: 892-897.

What is claimed is:

1. A method for preparing a cDNA sample for sequencing comprising the steps of:
creating a double stranded cDNA by annealing a RNA having a poly A tail with a Cap-Trsa-CV oligonucleotide and reverse transcribing the RNA resulting in a full length double stranded cDNA;
fragmenting the full length double stranded cDNA to generate a plurality of fragmented double stranded cDNA;
ligating a double stranded A+ adapter or a double stranded B+ adapter to a first end of each fragmented double stranded cDNA and a double stranded A+ adapter or a double stranded B+ adapter to a second end of each fragmented double stranded DNA using a ligase; and
amplifying the ligated double stranded cDNA using an amplification mixture comprising a primer A, a primer B, a A+-cap primer and a DNA polymerase.

2. The method of claim 1, wherein the Cap-Trsa-CV oligonucleotide comprise a cap primer sequence at the 5′ end and a broken poly T stretch region at the 3′ end, wherein the broken poly T stretch region comprises two poly T regions separated by at least one base residue selected from dA, dC, and dG.

3. The method of claim 1, wherein the Cap-Trsa-CV oligonucleotide comprises SEQ. ID. NO: 1.

4. The method of claim 2, wherein the cap primer sequence comprises SEQ ID NO. 2.

5. The method of claim 1, wherein the A+-cap primer comprises SEQ. ID. NO: 3.

6. The method of claim 1, wherein the A+ adapter comprises:
an A+ long oligonucleotide having a first suppression tag at the 3′ end and an A+ primer sequence at the 5′ end; and
an A+ short oligonucleotide comprises oligonucleotide complementary to the first suppression tag.

7. The method of claim 1, wherein the B+ adapter comprises:
a B+ long oligonucleotide having a second suppression tag at the 3′ end and a B+ primer region at the 5′ end; and
a B+ short oligonucleotide comprises oligonucleotide complementary to the second suppression tag.

8. The method of claim 1, wherein the B+ long oligonucleotide further comprises a bar-coding tag.

9. The method of claim 8, wherein the bar-coding tag comprises biotin.

10. The method of claim 1, wherein the step of fragmentation uses sonication.

11. The method of claim 1, wherein the step of ligation uses a molar ratio between about 0.9 to about 1.1 for the A+ adapter to B+ adapter.

12. The method of claim 1, wherein the step of amplification uses a molar ratio of between about 0.9-1.1 to about 1 for the primer A:primer B to A+-cap primer.

13. The method of claim 1, further comprising the step of amplifying the 5′ end separately with an Atitn primer, a Btitn primer and a halfswitch.

14. The method of claim 1, further comprising the step of amplifying the 3′ end separately with an Atitn primer, a Btitn primer and a TrsaC.

15. The method of claim 1, further comprising the step of amplifying the internal fragments with an Atitn primer and a Btitn primer.

16. A pair of adapter oligonucleotides for amplification comprising:
an A+ adapter and a B+ adapter, each comprising a long strand and an short strand and wherein each is capable of ligating to a first end or a second end of a fragmentated double stranded cDNA, wherein:
the long strand of the A+ adapter comprises an A primer region at the 5′ end and a first suppression tag region at the 3′ end, and
the long strand of the B+ adapter comprises a B primer region at the 5′ end and a second suppression tag region at the 3′ end, and
each of the first and second suppression tag regions cause a PCR suppression effect of the double stranded cDNA with A+ adapter and the double stranded cDNA with B+ adapter; and
the combination of the A+ adapter, the B+ adapter and a fragmented double stranded cDNA in the presence of a primer cocktail results in that only the double stranded cDNA with both A+ and B+ adapter are capable of being amplified.

17. The oligonucleotides of claim 16, wherein the primer cocktail comprises A primer, B primer, and A+-cap primer.

18. The oligonucleotides of claim 16, wherein the first and second suppression tag regions comprise the same sequence.

19. The oligonucleotides of claim 16, wherein either the long strand of the A+ or the B+adapter further comprises a bar-coding tag.

20. The oligonucleotides of claim 16, wherein either the long strand of the A+ or the B+ adapter further comprises a biotin tag.

21. The oligonucleotides of claim 16, wherein the long strand A+ adapter is selected from SEQ ID NO: 4 or NO: 5.

22. The oligonucleotides of claim 16, wherein the long strand B+ adapter is selected from SEQ ID NO: 6 or 7.

23. The oligonucleotides of claim 17, wherein the A+-cap primer comprises SEQ. ID NO: 3

24. The pair of adapter double strand oligonucleotide of claim 16, wherein the molar ratios of the A+ adapter:B+ adapter during the ligation step comprises about 0.9 to 1.1:about 0.9 to 1.1.

25. The pair of adapter double strand oligonucleotide of claim 17, wherein the molar ratio of A primer, B primer, and A+-cap primer comprises about 0.9 to 1.1:about 0.9 to 1.1:about 0.09 to 0.11.

26. A method for preparing a cDNA sample for sequencing comprising the steps of:
creating a double stranded cDNA by annealing a RNA having a poly A tail with a Cap-Trsa-CV oligonucleotide and reverse transcribing the RNA resulting in a full length double stranded cDNA;
fragmenting the full length double stranded cDNA to generate a plurality of fragmented double stranded cDNA;
ligating a double stranded A+ adapter or a double stranded B+ adapter to a first end of each fragmented double stranded cDNA and a double stranded A+ adapter or a double stranded B+ adapter to a second end of each fragmented double stranded DNA using a ligase; and
amplifying the ligated double stranded cDNA using an amplification mixture comprising a primer A, a primer B, a A+-cap primer, a Btitn-halfswitch primer and a Btitn-TrsaC primer and a DNA polymerase.


SEQUENCE LISTING (26 sequences)

SEQ ID NO: 1 - DNA, 49 bp, Artificial Sequence
  DNA Primer
aagcagtggtatcaacgcagagtcgcagtcggtacttttttcttttttv

SEQ ID NO: 2 - DNA, 23 bp, Artificial Sequence
  DNA Primer
aagcagtggtatcaacgcagagt

SEQ ID NO: 3 - DNA, 52 bp, Artificial Sequence
  DNA Primer
gcctccctcgcgccatcagccgcgcaggtaagcagtggtatcaacgcagagt

SEQ ID NO: 4 - DNA, 29 bp, Artificial Sequence
  DNA Primer
gcctccctcgcgccatcagccgcgcaggt

SEQ ID NO: 5 - DNA, 10 bp, Artificial Sequence
  DNA Primer
acctgcgcgg

SEQ ID NO: 6 - DNA, 30 bp, Artificial Sequence
  DNA primer
gccttgccagcccgctcagacgagcggcca

SEQ ID NO: 7 - DNA, 11 bp, Artificial Sequence
  DNA Primer
tggccgctcgt

SEQ ID NO: 8 - DNA, 49 bp, Artificial Sequence; identical to SEQ ID NO: 1
  DNA Primer

SEQ ID NO: 9 - DNA, 32 bp, Artificial Sequence
  Long Oligo
tccctgcgtgtctccgactcagccgcgcaggt

SEQ ID NO: 10 - DNA, 10 bp, Artificial Sequence
  Short Oligo
acctgcgcgg

SEQ ID NO: 11 - DNA, 42 bp, Artificial Sequence
  Oligo
tccctgcgtgtctccgactcagccgcggacgtacgtccgcgg

SEQ ID NO: 12 - DNA, 42 bp, Artificial Sequence
  Oligos
tccctgcgtgtctccgactcagccgcgagcgtacgctcgcgg

SEQ ID NO: 13 - DNA, 42 bp, Artificial Sequence
  Oligos
tccctgcgtgtctccgactcagccgcgcgagtactcgcgcgg

SEQ ID NO: 14 - DNA, 42 bp, Artificial Sequence
  Oligos
tccctgcgtgtctccgactcagccgcgacggtaccgtcgcgg

SEQ ID NO: 15 - DNA, 42 bp, Artificial Sequence
  Oligos
tccctgcgtgtctccgactcagccgcggcagtactgccgcgg

SEQ ID NO: 16 - DNA, 42 bp, Artificial Sequence
  Oligos
tccctgcgtgtctccgactcagccgcgctggtaccagcgcgg

SEQ ID NO: 17 - DNA, 42 bp, Artificial Sequence
  Oligos
tccctgcgtgtctccgactcagccgcgcgtgtacacgcgcgg

SEQ ID NO: 18 - DNA, 42 bp, Artificial Sequence
  Oligos
tccctgcgtgtctccgactcagccgcggtcgtacgaccgcgg

SEQ ID NO: 19 - DNA, 42 bp, Artificial Sequence
  Oligos
tccctgcgtgtctccgactcagccgcggctgtacagccgcgg

SEQ ID NO: 20 - DNA, 32 bp, Artificial Sequence
  Long Oligo
tgtgtgccttggcagtctcagacgagcggcca

SEQ ID NO: 21 - DNA, 11 bp, Artificial Sequence
  Short Oligo
tggccgctcgt

SEQ ID NO: 22 - DNA, 22 bp, Artificial Sequence
  DNA Primer
tccctgcgtgtctccgactcag

SEQ ID NO: 23 - DNA, 21 bp, Artificial Sequence
  DNA Primer
tgtgtgccttggcagtctcag

SEQ ID NO: 24 - DNA, 53 bp, Artificial Sequence
  DNA Primer
tgtgtgccttggcagtctcagacgagcggccagtatcaacgcagagtacatgg

SEQ ID NO: 25 - DNA, 57 bp, Artificial Sequence
  DNA primer
tgtgtgccttggcagtctcagacgagcggccacgcagtcggtacttttttctttttt

SEQ ID NO: 26 - DNA, 44 bp, Artificial Sequence
  DNA Primer
agtggactatccatgaacgcaaagcagtggtatcaacgcagagt